(19) United States
(12) Patent Application Publication
(10) Pub. No.: US 2026/0243636 A1
(43) Pub. Date: Aug. 20, 2026

(54) DIAGNOSTIC SYSTEM AND METHOD

(71) Applicant: NGB INNOVATION PTY LTD, Blakehurst (AU)

(72) Inventor: Nan ZHANG, Blakehurst (AU)

(21) Appl. No.: 19/491,003

(22) PCT Filed: May 24, 2024
(86) PCT No.: PCT/AU2024/050536

§ 371 Date: Dec. 8, 2025

(30) Foreign Application Priority Data
May 26, 2023 (AU) 2023901648

(51) Int. Cl. G01N 1/40; B01F 33/452; B01F 33/453; C12N 1/066; C12N 13/00; C12Q 1/6806

(52) U.S. Cl. CPC G01N 1/40; B01F 33/452; B01F 33/4534; C12N 1/066; C12N 13/00; C12Q 1/6806

(57) ABSTRACT

The present disclosure is directed to a cell lysis systems and methods for preparing a sample for analysis. A sample to be analysed is prepared by introducing the sample and a reagent mixture into a sample tube together with an agitator, and agitating the sample in the sample tube such that cells in the sample undergo lysis.

### TECHNICAL FIELD

The present invention is directed to diagnostic systems and methods. In particular, the present invention is directed to systems and methods for the preparation of samples using cell lysis for the purposes of diagnostic testing.

### BACKGROUND

In vitro diagnostic devices are the front line to detect the presence of harmful pathogens within the environment and/or objects. Such devices are an important tool in reducing the possibility of the spread of the virus between animals, animal to human and human to human. In addition, such devices can assist in the analysis and identification of biomarkers associated with illness and disease.

COVID-19 is a respiratory illness that is caused by the novel SARS-CoV-2. SARS-CoV- 2 was first known to infect people in 2019 leading to a global pandemic, triggering severe social and economic disruption around the world.

Testing for SARS-CoV-2 is primarily undertaken by either detecting the presence of the virus's inner RNA (e.g. nucleic acid tests), or by detecting the presence of antibodies against the spike (S) protein and/or detecting antibodies against the nucleocapsid (N) protein. (e.g. rapid antigen test (RAT)).

There are different types of nucleic acid tests that can be used to detect SARS-CoV-2 viral RNA, including real-time quantitative reverse transcription-PCR (rqRT-PCR) and isothermal nucleic acid amplification tests (e.g. loop-mediated isothermal amplification (LAMP) tests).

PCR tests are generally considered better at detecting the presence of the SARS-CoV-2 virus and are currently the gold standard for diagnosis of COVID-19.

PCR assays typically take several hours (including specimen processing time) to generate results and require complex laboratory equipment and trained technicians.

Rapid antigen tests (RATs), using lateral flow method or other methods, can provide faster test results (typically 5 to 30 minutes) and do not require a trained professional for administration. However this method is considered less sensitive than PCR testing, with the accuracy of this method being limited by the number of available target analytes, sample type, and virus load.

One option for improving the accuracy of such tests is through improved cell lysis of a sample to be tested, thus increasing the biomarkers in the sample to be tested.

Cell lysis is the breaking down and/or maximum loosening of the membrane of a cell in order to release its contents. Cell lysis may be achieved in many ways, including by chemical, acoustic, viral, enzymatic, osmotic, or mechanical mechanisms.

Cell lysis provides a lysate, a fluid containing the contents of the lysed cell, which can then be used to purify or further study the cell contents. By breaking the cell membrane, more biomarkers from the sample can be released, thus improving the accuracy of the results.

However, existing cell lysis devices are bulky, require relatively more samples. In addition, existing devices are not necessarily suitable for sensitive testing and can expose samples to potential contamination. Existing cell lysis reagents are limited by the strain and type of pathogens and the nature of the sample to be tested.

It is desirable to provide a sample preparation device that is portable, relatively cheap, does not allow for contamination between samples, is easy to use and facilitates accurate diagnostic results upon analysis and testing of the prepared sample.

### SUMMARY

According to one aspect of the present disclosure, there is provided a cell lysis system for preparing a sample for analysis, the system comprising:
a reagent mixture comprising at least two reagents having different molar masses;
a sample tube for receiving the sample to be prepared and the reagent mixture; an agitator element configured to be received within the sample tube, wherein the agitator element comprises magnetic material;
a drive generator for generating drive forces acting on the agitator element, the generated drive forces comprising magnetic forces spaced from and rotatable about a drive axis;
a sample tube holder for receiving the sample tube, wherein the sample tube holder is positioned such that rotational magnetic forces generated by the drive generator generates movement of the agitator element within the sample tube; and an agitator stabiliser configured to be positioned within the sample tube for stabilising the agitator element during movement generated by the drive generator.

According to a further aspect of the present disclosure, there is provided a method of preparing a sample for analysis with a system according to the present disclosure, the method comprising:
obtaining the sample to be prepared;
placing the sample to be prepared and the reagent mixture in the sample tube;
placing the agitator element in the sample tube;
placing the agitator stabiliser in the sample tube;
placing the sample tube containing the sample to be prepared, the reagent mixture, agitator element, and the agitator stabiliser in the sample tube holder;
operating the drive generator to generate magnetic forces rotating about the drive axis thereby to generate movement of the agitator element within the sample tube such that cells in the sample undergo lysis.

### BRIEF DESCRIPTION OF DRAWINGS

Embodiments of the disclosure will now be described by way of example only with reference to the accompanying drawings in which:

FIG. 1a shows a cutaway side view of a sample tube according to an embodiment of the present disclosure, the sample tube containing a sample;

FIG. 1b shows a cutaway side view of the sample tube of FIG. 1a with a spherical agitator and stabiliser inserted;

FIG. 1c shows the sample tube of FIGS. 1a and 1b in a sample tube holder of a cell lysis device according to and embodiment of the present disclosure;

FIG. 2a shows a side view of a sample tube with lid and agitator stabiliser according to another embodiment of the present disclosure;

FIG. 2b shows a side view of a sample tube with lid and agitator stabiliser according to a further embodiment of the present disclosure;

FIG. 3 shows a flowchart of a method of preparing a sample for analysis;

FIG. 4a shows ladders of samples prior to (A1) and after (B1, C1, D1, E1, F1, G1, H1, A2, B2, C2) processing;

FIG. 4b shows a size distribution profile from a sample (A1 of FIG. 4a) prior to processing in a system according to an embodiment of the present disclosure

FIG. 5a ladders of samples prior to (L1) and after (B1, C1, D1, E1, F1, G1,H1, A2, B2, C2) processing

FIG. 5b shows a size distribution profile from a sample (L1 of FIG. 5a) prior to processing in a system according to an embodiment of the present disclosure.

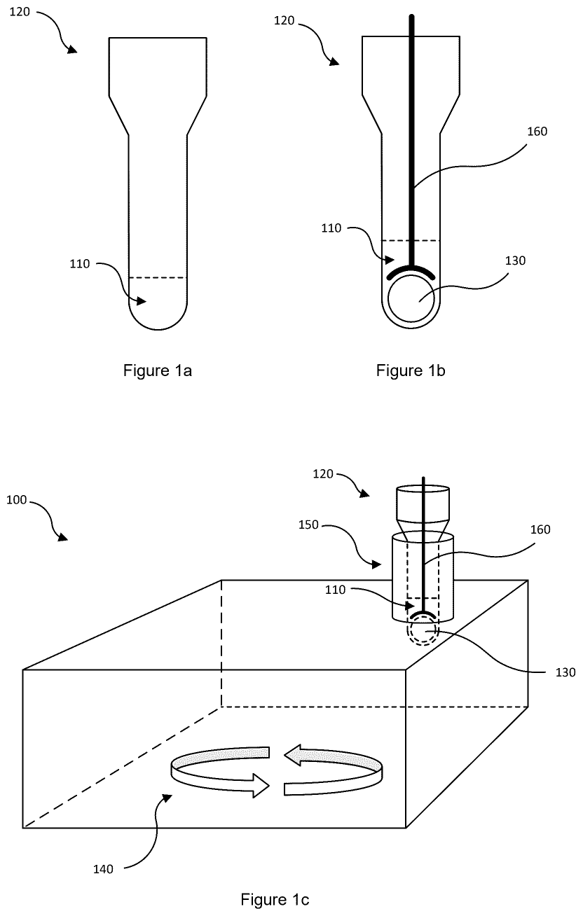

### DESCRIPTION OF EMBODIMENTS

According to the present disclosure, there is provided a cell lysis system 100 for preparing a sample for a diagnostic device. The system 100 will be described with regard to the preparation and analysis of biological samples for medical diagnoses and/or the detection of pathogens, however it will be appreciated that the described system may also be used for the preparation of samples in other fields.

The described system 100 need not be limited to human use, but may also be used with animal samples, for example in agriculture and farming. Alternatively, the system may be used to test manufactured consumer goods such as food, pharmaceuticals or cosmetics.

The sample to be prepared may be a biological sample such as saliva, blood or urine. Alternatively, the sample to be prepared may be sampled from a variety of surfaces or water samples. The sample to be prepared may be directly obtained, e.g. saliva, food or cosmetic samples. Alternatively the sample may be obtained through other means, such as swabbing or a blood draw. In an embodiment, the sample to be prepared is obtained via nasopharyngeal, throat or nasal swab. In another embodiment, the sample to be prepared is obtained by swabbing a surface to be tested.

The cell lysis system 100 comprises a reagent mixture 110 comprising at least two reagents having different molar masses.

The at least two reagents are at least partially immiscible. In an embodiment, at least two reagents have low miscibility..

The molar mass of each reagent is in the range of from 30 g/mol to 500 g/mol. For example, the molar mass of each reagent may be of about 30 g/mol, 40 g/mol, 50 g/mol, 60 g/mol, 70 g/mol, 80 g/mol, 90 g/mol, 100 g/mol, 110 g/mol, 120 g/mol, 130 g/mol, 140 g/mol, 150 g/mol, 160 g/mol, 170 g/mol, 180 g/mol, 190 g/mol, 200 g/mol, 210 g/mol, 220 g/mol, 230 g/mol, 240 g/mol, 250 g/mol, 260 g/mol, 270 g/mol, 280 g/mol, 290 g/mol, 300 g/mol, 310 g/mol, 320 g/mol, 330 g/mol, 340 g/mol, 350 g/mol, 360 g/mol, 370 g/mol, 380 g/mol, 390 g/mol, 400 g/mol, 410 g/mol, 420 g/mol, 430 g/mol, 440 g/mol, 450 g/mol, 460 g/mol, 470 g/mol, 480 g/mol, 490 g/mol, or 500 g/mol. The molar mass may be in a range between any two of these values.

The reagent mixture 110 may further comprise metal nanoparticles. The nanoparticles may assist in distinguishing analytes within the sample. In an embodiment, the reagent mixture 110 comprises iron oxide (Fe₃O₄). In another embodiment, the reagent mixture 110 comprises gold nanoparticles.

The reagent mixture 110 may further comprise a lysing reagent capable of breaking down the cell membrane. The lysing reagent may be a chemical lysing regent or an enzymatic lysing reagent. In one embodiment, the reagent mixture 110 comprises protease. Additionally or alternatively, the reagent mixture 110 may comprise one or more reagents selected to disrupt electrostatic interactions within the cell membrane to facilitate the lysing process. In another embodiment, the pH of the reagent mixture 110 may be selected to facilitate the lysing process.

In an embodiment, the reagent mixture 110 comprises a mixture of dimethylsulfoxide (DMSO) and water. Optionally, the reagent mixture may further comprise oil and/or ethanol.

The cell lysis system 100 comprises a sample tube 120 for receiving the sample to be prepared and the reagent mixture 110. The sample tube 120 may further comprise a removable lid 125 for sealing the contents within the tube.

In some embodiments, the sample tube 120 may be provided pre-filled with the reagent mixture 110 and into which the sample to be prepared is added. In other embodiments, the reagent mixture 110 may be provided separately for adding into the sample tube 120 at the time the sample is to be prepared. In still other embodiments, one or more reagent components for forming the reagent mixture 110 may be stored separately for adding to the sample tube 120 at the time the sample is to be prepared.

The cell lysis system 100 further comprises an agitator element 130 configured to be received within the sample tube 120. The agitator element 130 comprises magnetic material. The agitator element 130 may be formed in whole or in part by magnetic material.

The agitator element 130 is sized and shaped to fit within the sample tube 120 such that a layer of fluid may form between the surface of the agitator element 130 and the internal walls of the sample tube 120. In an example, the formed fluid layer may be around 10 mm. Preferably the bottom of the sample tube 120 has a complimentary shape to the shape of the agitator element 130.

Preferably, the agitator element 130 is spherical. In this embodiment, the bottom of the sample tube 120 is hemi-spherical or semi-spherical.

The cell lysis system 100 further comprises a drive generator 140 for generating drive forces acting on the agitator element 130. The generated drive forces comprising magnetic forces spaced from and rotatable about a drive axis.

In an embodiment, the drive generator 140 comprises one or more drive magnets spaced from and rotatable about the drive axis. Alternatively, the drive generator 140 comprises an electromagnetic source for generating the magnetic forces.

A sample tube holder 150 is further provided for receiving the sample tube 120 thereby to position the sample tube 120 relative to the drive generator 140. The sample tube holder 150 is positioned such that the rotation of the magnetic forces about the drive axis generates movement of the agitator element 130 within the sample tube 120 The drive generator 140 is configured to rotate at sufficient speed such that the resulting movement of the agitator element 130 results in shear forces generated by the at least two reagents to adequately break down the sample for analysis or diagnostic testing. In an embodiment, the drive generator 140 operates to generate rotation of the magnetic forces at a speed of from 3,000 rpm to 20,000 rpm.

Movement of the agitator element 130 comprises rotation of the agitator element 130 about one or more axes. Additionally, movement of the agitator element 130 comprises movement of the agitator element 130 along one or more axes. The resulting movement is a combination of rotational and vibrational movements.

In some embodiments, operation of the drive generator 140 is battery powered.

The system further comprises an agitator stabiliser 160 configured to be positioned within sample tube 120 for stabilising the agitator element 130 during movement generated by the drive generator 140. Preferably, the agitator stabiliser 160 has a complimentary shape to the agitator element 130. More preferably, the combination of the end of the sample tube 120 and the agitator stabiliser 160 co-operate to substantially encase the agitator element 130 in the sample tube 120.

The agitator stabiliser 160 may be formed of a swab used to obtain a sample to be prepared. Alternatively, the agitator stabiliser 160 may be a separate insert for positioning in the sample tube 120. In one embodiment, the agitator stabiliser 160 is attached to the sample tube lid 125.

Movement of the agitator element 130 causes fluid friction between the agitator element 130 and the two or more reagents forming the reagent mixture 110. As the agitator element 130 is sized to fit closely within the sample tube 120, a layer of turbulent flow forms between the agitator element 130 and the sample tube 120 inner wall, with the different molar mass of the reagents moving at different speeds and assisting in generating the shear force within the sample tube 120 sufficient to lyse the cells in the sample to be prepared. This may be further facilitated by providing a mixture comprising a polar reagent and a non-polar reagent, whereby the polar and non-polar reagents, which are rotating at different speeds, target different parts of the cell membrane, further assisting in the breakdown of the membrane.

During operation, temperatures of from 50° C. to 120° C. may be generated within the sample tube created by the various generated forces, such as friction between the surfaces of the magnetic ball 130 and the inner wall of the sample tube 120.

During operation, pressures of up to 2 kPa may be generated within the sample tube 120.

The described system is designed to be portable and easy to use, and available for widespread use without requiring highly trained technicians in order to prepare samples. By way of example, it is envisaged that such a system may be used, e.g. to prepare a sample for use with RATs, at home, pharmacies, medical clinics, retail settings, laboratories or hospitals.

Although the system has been described with regard to preparation of samples for SARS-CoV-2 testing, it will be appreciated that the system may be used for the preparation of samples for diagnostic testing of a variety of infections (e.g. monkeypox, HIV, Zika), pathogens (e.g. E. coli, Salmonella, foot and mouth disease, Aflatoxin, rabies) and other diseases (e.g. cancer, chronic inflammatory disease). Although the system has been described with respect to cell lysis applications, it will be appreciated that the system may be used for other applications, for example gene cutting applications, and in particular preparing DNA and/or RNA fragments of similar size.

### EXAMPLES

### Example Test Protocol

An example testing protocol comprises:
Wash hands before the test.
Remove the sample collector from the packet.
Rub the collector tip against the tongue and the inside of both cheeks for 30 sec.
Open the sample tube comprising the reagent mixture.
Insert the sample collector into the sample tube.
Remove the magnetic ball from the packet. Note: directly push the ball into the sample tube. Do not touch the ball.
Close the lid of the tube.
Place sample tube in sample tube holder.
Turn on the device for 2 minutes.
Using a syringe, transfer the processed sample to the RAT cartridge.
Read the results within 5 minutes.

### SARS-CoV-2Test Example

A SARS-CoV-2 positive sample was prepared using a method according to the present disclosure with a device rotation speed of 2000-2500 rpm for approximately 2-5 minutes, chitosan and sodium chloride 0.9% as reagents, and a ratio 2 chitosan: 1 sodium chloride. The prepared sample was tested using a SARS-CoV-2 Antigen Rapid Test Cassette from Biohit Healthcare (Hefei). The same sample without additional preparation was also tested using a SARS-CoV-2 Antigen Rapid Test Cassette from Biohit Healthcare (Hefei). A PCR test confirmed the individual's positive status.

A negative RAT results was obtained in the sample without further processing. The sample processed using the method according to the present disclosure produced a clear positive line.

### Gene Cutting

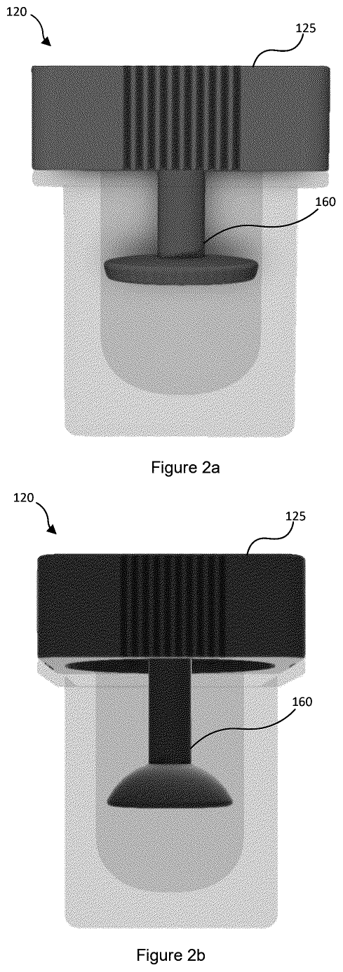

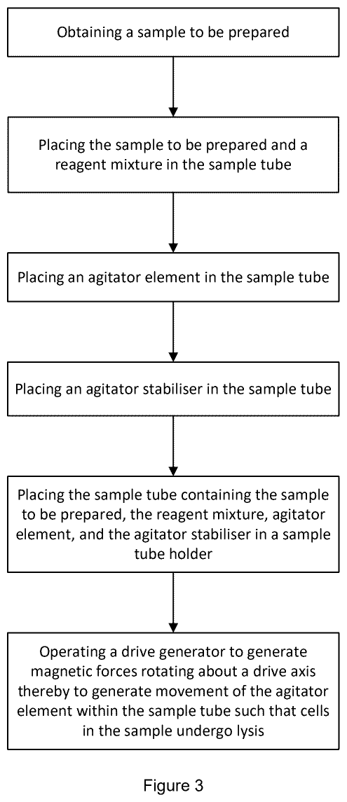

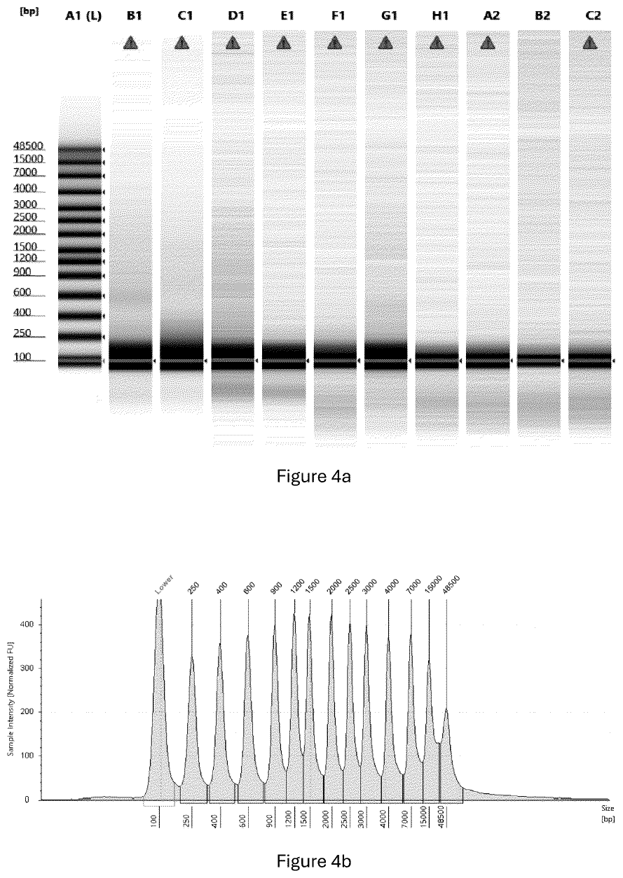

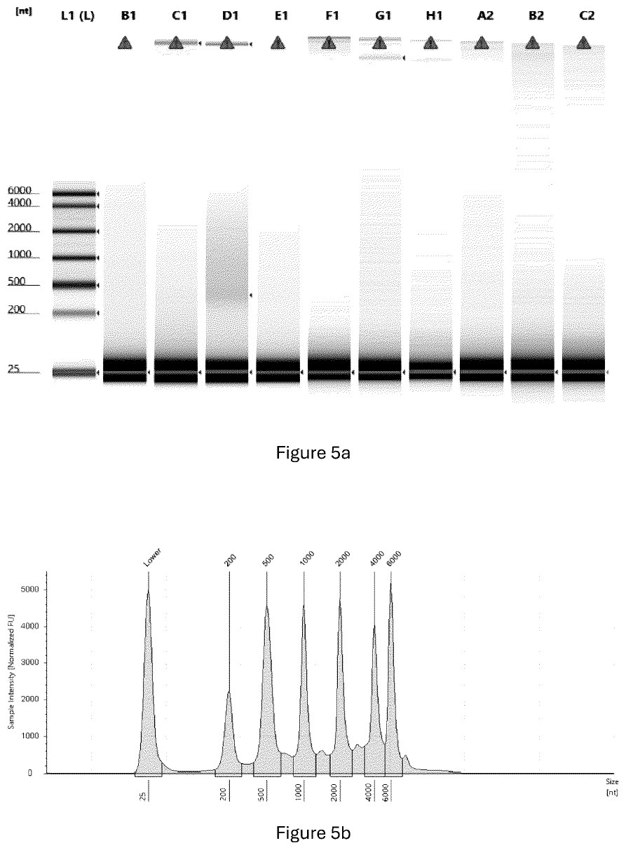

Additional testing was conducted using sample tubes in accordance with the embodiments shown in FIGS. 2a and 2b with a rotation speed of 5000 rpm and with a reagent mixture comprising dimethylsulfoxide (DMSO). The results of the processing are shown in particular in FIGS. 4a and 5a in which it can be seen the original sample was reduced to smaller fragments. In contrast to traditional chemical and mechanical methods which produce DNA and RNA fragments of varying size, the present system results in fragments of a similar size.

It will be appreciated a sample may undergo both cell lysis and gene cutting in a single step, in which the sample is treated to break down the cell membranes and release the DNA and RNA chains or organelles containing DNA and RNA chains to be released into the reagent mixture. The vibration and heat produced within the system can then be utilised to break the DNA and RNA chains.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A cell lysis system for preparing a sample for analysis, the system comprising:
a reagent mixture comprising at least two reagents having different molar masses;
a sample tube for receiving the sample to be prepared and the reagent mixture;
an agitator element configured to be received within the sample tube, wherein the agitator element comprises magnetic material;
a drive generator for generating drive forces acting on the agitator element, the generated drive forces comprising magnetic forces spaced from and rotatable about a drive axis;
a sample tube holder for receiving the sample tube, wherein the sample tube holder is positioned such that rotational magnetic forces generated by the drive generator generates movement of the agitator element within the sample tube; and
an agitator stabiliser configured to be positioned within the sample tube for stabilising the agitator element during movement generated by the drive generator.

2. A system according to claim 1, wherein the at least two reagents have low miscibility.

3. A system according to claim 1, wherein the molar mass of each reagent is in the range of from 30 g/mol to 500 g/mol.

4. A system according to claim 1, wherein the reagent mixture comprises dimethylsulfoxide (DMSO) and water.

5. A system according to claim 4, wherein the reagent mixture further comprises oil and/or ethanol.

6. A system according to claim 4, wherein the reagent mixture further comprises metal nanoparticles.

7. A system according to claim 1, wherein the agitator element is sized to form a fluid layer of less than around 10 mm between an outer surface of the agitator element and an internal surface of the sample

8. A system according to claim 1, wherein the agitator element is spherical.

9. A system according to claim 1. wherein the drive generator comprises one or more drive magnets spaced from and rotatable about the drive axis.

10. A system according to claim 9, wherein the one or more drive magnets rotate at a speed of from 3,000 rpm to 20,000 rpm.

11. A system according to claim 1, wherein the drive generator comprises an electromagnetic source for generating the magnetic forces.

12. A method of preparing a sample for analysis with a system according to claim 1, the method comprising:
obtaining the sample to be prepared;
placing the sample to be prepared and the reagent mixture in the sample tube;
placing the agitator element in the sample tube;
placing the agitator stabiliser in the sample tube;
placing the sample tube containing the sample to be prepared, the reagent mixture, agitator element, and the agitator stabiliser in the sample tube holder;
operating the drive generator to generate magnetic forces rotating about the drive axis thereby to generate movement of the agitator element within the sample tube such that cells in the sample undergo lysis.
